(19) United States
(12) Patent Application Publication
(10) Pub. No.: US 2001/0008638 A1
(43) Pub. Date: Jul. 19, 2001

(54) NUTRITIONAL SUPPLEMENT COMPRISING AT LEAST ONE PROHORMONE AND AT LEAST ONE ANTI-ESTROGEN COMPOUND IN A TIME-RELEASE DOSAGE FORM

(76) Inventor: BRIAN J. WILDING, WINNIPEG (CA)

Correspondence Address: STANDLEY & GILCREST LLP, 495 METRO PLACE SOUTH, SUITE 210, DUBLIN, OH, 43017, US

(21) Appl. No.: 09/352,084

(22) Filed: Jul. 14, 1999

(51) Int. Cl. A61K 31/56; A61K 9/00; A61K 9/52; A61K 9/22; A61K 9/28

(52) U.S. Cl. 424/468; 424/474; 424/457; 514/178; 514/177; 514/182

(57) ABSTRACT

The present invention relates to an oral dosage delivery form which is effective in increasing the testosterone levels in humans. In a preferred embodiment, the composition comprises a mixture of four (4) prohormones, three (3) anti-estrogen agents and at least one (1) enteric coating. The inventive prohormone/anti-estrogen time-release dosage form allows the prohormone/anti-estrogen ingredients to be released over an extended period of time so as to provide for a sustained elevated blood serum testosterone level.

### TECHNICAL FIELD

This invention relates to a nutritional supplement that comprises at least one (1) anabolic compound, such as 4-androstene-3β, 17β diol and at least one (1) anti-estrogen compound, such as saw palmetto extract, in a time-release dosage form that, for example, comprises at least one (1) enteric coating, more preferably at least two (2) enteric coatings.

Background of the Invention

The steroid hormone testosterone is considered to be the male virility hormone. Its effects include maintenance of muscle and bone mass, sexual function and physiological well being. After about the age of 35, the typical human male experiences a slow decline in testosterone levels. This decline in testosterone levels results in depression, lethargy, lack of sexual desire and loss of muscle mass and strength.

Testosterone injections, in oil depot form, have been used to counteract the decline in testosterone levels, however, these injections are inconvenient, often painful and result in inconsistent blood levels. Typically, a supraphysiological surge is seen immediately after the injection, but by the time the next injection is due, the levels have often dropped below their standard physiological level. This is in contrast to the typical cycle of testosterone in human males which pulses about every 90 minutes. The extremely high blood serum levels of testosterone found after depot injections may cause prostrate hypertrophy as well as the potential shutdown of the hypothalamic/pituitary testicular axis.

An alternative to testosterone injections is the oral administration of androgen derivatives. The enteral consumption of compounds such as methyltestosterone and fluoxymesterone have been found to increase the blood serum levels of testosterone. These compounds have been altered in the 17α position of the steroid molecule through the addition of an alkyl group. The alkyl group renders the steroid impervious to oxidation of the 17β hydroxyl group in the liver and thereby greatly improves its oral bioavailability compared to non-alkylated steroids. It has been reported however, that this structural modification is associated with a greatly increased risk of liver toxicity. Therefore, these compounds are not readily acceptable in combating a decrease in testosterone levels in the human male.

While increased levels of testosterone in human males may have many positive attributes, it is also known that there are adverse side effects. Certain enzymes in the body aromatize testosterone to estrogen, thus reducing the level of testosterone. The increased level of estrogen causes the body to produce fat and deposit it as adipose tissue. Further, prolonged increased estrogen levels can cause men to develop gynecomastia.

Testosterone is also converted to dihydrotestosterone (DHT) by the enzyme 5-α reductase. It has been postulated that DHT is responsible for benign prostatic hyperplasia (BHP). DHT is about ten (10) times more potent than testosterone and an abnormally high amount of testosterone in prostrate tissue results in it being converted to DHT. These high levels of DHT trigger cellular growth leading to enlargement of the prostrate gland. DHT also increases mitochondrial activity thereby creating more oxidative stress.

There are numerous compounds that are known to be anti-estrogenic. As used herein and in the claims, the term “anti-estrogen compound” means a compound that will decrease the conversion of testosterone to estrogen and/or DHT. One such anti-estrogen compound is an extract from the berries of the saw palmetto plant (Serenoa Repens). Saw palmetto is a small palm tree with large leaves and large, deep, red-blackberries. The berries were used by the American Indians as a general tonic to nourish the body and encourage appetite and normal weight gain. Saw palmetto berries contain an oil with a variety of fatty acids and phytosterols. These fatty acids include capric, caprylic, caproic, lauric, palmitic and oleic acid and their ethyl esters. The major phytosterols are β-sitosterol, stig masterol, cycloartenol, stigmas terol, lupeol, lupenone and 24-methyl-cycloartenol. The fat soluble extract of saw palmetto berries has been shown to inhibit the conversion of testosterone to DHT. As discussed above, DHT is thought to be responsible for the enlargement of the prostrate.

Another compound known to have anti-estrogen properties is chrysin. Chyrsin (5,7-dihydroxyflavone) is a bioflavonoid. Chyrsin is isolated from Passiflora plants such as P. coerulea and P. incarnate (also known as passion flower). The additional effects of chrysin include anti-inflammatory action, anti-viral, vasodilator and anxiety reducing properties. It is believed that this bioflavinoid interferes with the aromatization of testosterone to estrogen.

Another phytochemical known to have anti-estrogen properties is indole-3-carbinol. Indole-3-carbinol is found in cabbage, broccoli and brussel sprouts and it has been shown, in pre-clinical experiments, to be a promising nutrient. In fact, a considerable body of pre-clinical evidence has been accumulated on the efficacy of indole-3-carbinol in the prevention and intervention of direct and indirect acting carcinogens in the development of breast tumors in rodents. One uncertainty relates to what is specifically responsible for the anti-estrogen effects of indole-3-carbinol, the chemical itself or one of the compounds it is converted to when it contacts the acids in the stomach or when it reacts with other chemicals in the cell.

In the traditional medicine of the Orient and Bulgaria, the plant Tribulus terrestris L. from the Zigofilaceae family has long been known as a medicinal plant for the treatment of a number of diseases and most often for spermatorrhea and sexual impotence. The non-hormonal extract from Tribulus terrestris is a phytochemical and, in clinical studies has been shown to produce a generally tonic effect and a marked effect upon the sexual system. The extract is prepared from the overground part of the plant (commonly known as Puncture Vine) and contains mainly steroid saponins of the furostanol type with a predominant quantity of protodioscin. While Tribulus terrestris is a phytochemical and technically not a prohormone, for purposes of this application and the claims herein, it shall be classified as a prohormone.

It is an aspect of the present invention that a preferred combination of effective anti-estrogen ingredients such as chrysin, indole-3-carbinol and saw palmetto extract be combined with a mixture of prohormones such as 19 Nor-4-androstene-3,17 dione; 4-androstene-3β, 17β diol; 5-androstene-3β, 17β diol; and Tribulus Terrestris. More importantly, the present invention is directed to a time-release oral dosage form wherein the mixture of prohormones and anti-estrogen ingredients are preferably encapsulated in at least one (1), preferably at least two (2), and most preferably three (3) enteric coatings. These enteric coatings provide for the time-release of the active components so that the blood serum levels of testosterone are consistent over a long period of time.

Background Art

U.S. Pat. No. 5,880,117 to Arnold discloses a method of increasing the testosterone levels in humans through the administration of 4-androstenediol. This patent teaches that testosterone levels in humans can be increased through the enteral administration of from 25 to 500 mg per day of 4-androstenediol. This patent also discloses that the preferred 4-androstenediol is 4-androstene-3β, 17β diol.

U.S. Pat. No. 5,578,588 to Mattern et al. discloses a method of increasing testosterone levels in humans by administering a testosterone precursor, namely androstenedione. Modes of administration discussed include parenteral, enteral and intra-nasal. Androstenedione is a natural steroid hormone found in the blood and are not 17α alkylated compounds and thus, hepatoxicity is minimized.

One aspect of the present invention relates to the inclusion of the inventive prohormone/anti-estrogen mixture in a time-release or controlled release dosage form. As used herein and in the claims, the terms “time-release” and “controlled release” means dosage forms (i.e., capsules, tablets, pills, pellets, granules and the like) that release the inventive prohormone/anti-estrogen mixture over a period of time in the human digestive system so as to result in a blood serum level of active ingredients that is more consistent over a six (6) to eight (8) hour period of time than the same dose of actives delivered in a non-time-release or non-controlled release dosage form. While the present invention has preferred methodologies of achieving the time-release feature, other known types of time-release dosage forms are contemplated herein. The following patents disclose various technologies that can be used to accomplish the time-release feature of the present invention.

U.S. Pat. No. 4,968,508 to Oren et al. relates to a matrix composition for sustained drug delivery which is comprised of an active agent, a hydrophilic polymer and an enteric polymer. The enteric polymer is impervious to gastric fluids and aids in retarding drug release in regions of low pH, thus allowing lower levels of hydrophilic polymer to be employed. Oren et al. suggests that this approach is useful in sustaining the release of numerous active agents whose solubility declines as the pH is increased. This patent does not suggest nor disclose the division of a combination of prohormones and anti-estrogen ingredients by two (2) or more enteric coatings.

U.S. Pat. No. 4,994,260 to Kallstrand et al. relates to a pharmaceutical preparation for controlled release of a pharmaceutically active substance prepared by mixing, in an aqueous carrier, a pharmaceutically active substance encapsulated in a coating and 60 to 99% by weight of a release controlling substance selected from the group consisting of polysaccharides, oligosaccharides, disaccharides, monosaccharides, polyhydroxyl alcohols and mixtures thereof. This patent describes the use of Eudragit® E-100 and sucrose to make the dosage form.

U.S. Pat. No. 5,188,836 to Muhammad et al. discloses a semi-enteric, sustained release pharmaceutical consisting of a biologically active composition layered on an inert core and an outer inert coating consisting of a water insoluble methacrylic acid polymer, a water soluble sugar alcohol, a food grade acid and a plasticizer characterized by a two-tiered solubility profile in the human digestive tract. The dosage forms of this reference initially dissolve in the stomach and thereafter completely dissolves and is absorbed in the intestine. This patent discloses the use of Eudragit® L30D as a major coating constituent.

U.S. Pat. No. 5,238,686 to Eichel et al. discloses a dual walled coated medicament having a water soluble core drug, an inner wall microencapsular coating and an outer wall enteric coating. By enterically coating the microcapsules, the release of core drug into the stomach is greatly impeded and the delivery of the drug is substantially delayed until the coated microcapsules reach the intestine.

Great Britain Patent No. 1147245 to Corvi-Mora relates to a slow release dosage form made up of many discrete particles having a maximum dimension of 2 mm. The particles are formed by compression of a powder consisting of the pharmaceutically active material and an inert carrier on a multiple-die tableting machine. The tablets produced are transferred to a copper pan and shaken to improve their spherical form. The tablets are then sprayed with a coating material which will dissolve slowly in gastric juices. Finally, the tablets are encapsulated in a gelatin capsule.

U.S. Pat. No. 3,857,933 to Ross et al. relates to a controlled release, therapeutically active ingredient wherein the dosage form comprises a capsule filled solely with beads of an average diameter of from about 0.1 to about 2.5 mm. Each bead consists of a castable carrier which is sparingly soluble in the digestive tract, which may be a glycol ester of a wax acid containing from 22 to 39 carbon atoms. The beads also contain up to 30% of the active ingredient and from 5 to about 22% of a physiologically tolerated surface-active substance.

Other references disclosing various methodologies of preparing compositions for controlled availability include U.S. Pat. No. 4,153,682 to Acker et al.; U.S. Pat. No. 4,542,011 to Gleixner et al.; U.S. Pat. No. 4,786,508 to Ghebre-Sellassie et al.; U.S. Pat. No. 4,980,170 to Schneider et al.; U.S. Pat. No. 5,382,601 to Nurnberg et al.; U.S. Pat. No. 5,476,667 to Kristensen et al.; and U.S. Pat. No. 5,707,655 to Kanikanti et al.

Disclosure of the Invention

During the course of his research, the inventor was concerned that increases in the blood testosterone levels seen with the oral administration of androstenedione, in a non-time-release dosage form, were erratic and different from the natural occurring pattern in the human male. It is therefore an aspect of the present invention that naturally occurring testosterone precursors be provided in such a manner that increased and sustained blood testosterone levels can be realized. It is an additional aspect of the present invention that at least one testosterone prohormone selected from the group consisting of 19 Nor-4-androstene-3,17 dione; 4-androstene-3β, 17β diol; 5-androstene 3β, 17β diol; and Tribulus Terrestris and mixtures thereof, be combined with at least one anti-estrogen ingredient selected from chrysin, indole-3-carbinol, saw palmetto extract and mixtures thereof. It is a further aspect of the present invention that this mixture of prohormones and anti-estrogen ingredients be administered in a time-release or controlled release dosage form that comprises at least one (1) enteric coating.

In a preferred embodiment, the composition of the present invention comprises all four (4) prohormones in combination with all three (3) anti-estrogen ingredients. However, it should be understood that all various permutations of any one given prohormone with one or more of the anti-estrogen ingredients are within the scope of the present invention. In the most preferred embodiment, all prohormones and all anti-estrogen ingredients are administered in a dosage form that comprises at least three (3), more preferably at least four (4), and most preferably at least five (5) enteric coatings.

In a yet more preferred embodiment, the present invention provides to a human in need of increasing his blood serum testosterone level, in a time release dosage form, the following compounds at the recited ranges:

| | | |
|---|---|---|
| a) | 19 Nor-4-androstene 3,17 dione | 25-200 mg |
| b) | 4 Androstene-3,17 diol | 25-200 mg |
| c) | 5 Androstene-3,17 diol | 10-100 mg |
| d) | Tribulus terrestris | |
| | (at least 35% steroidal saponins) | 50-500 mg |
| e) | Chrysin | 30-300 mg |
| f) | Indole-3-carbinol | 10-100 mg |
| g) | Saw palmetto extract | 40-400 mg |

In still a more preferred embodiment, each dosage form according to the invention contains about:

50 mgs of 19 Nor-4-androstene 3,17 dione

50 mgs of 4 Androstene-3,17 diol

25 mgs of 5 Androstene-3,17 diol

125 mgs of Tribulus terrestris (at least 35% steroidal saponins)

75 mgs of Chrysin

25 mgs of Indole-3-carbinol; and

90 mgs of saw palmetto extract.

An additional embodiment of the present invention relates to a method of increasing testosterone levels in humans through the administration of the prohormone/anti-estrogen composition described above in a time-release dosage form. In one embodiment, the method comprises administering the formula disclosed above in a time-release dosage form that is prepared through a wet granulation process wherein an aqueous solution of an enteric coating material is used to prepare the granules. Through the use of the composition and dosage form of the present invention, the anabolic/anti-estrogen ingredients are allowed to be released in a time-release fashion (i.e., over a six (6) hour period) to sustain elevated blood serum testosterone levels.

Best Mode for Carrying Out the Invention

As used herein and in the claims, the chemical term “4-androstenediol” refers to two isomers, 4-androstene-3β, 17β diol and 4-androstene-3α, 17β diol. 4-androstenediol is a naturally occurring compound. It has been identified as a metabolite of testosterone in placenta, testicular adrenal and hypothalmic/pituitary tissues. It acts as a very effective precursor to testosterone. 4-androstenediol converts to testosterone via the 3β-hydroxysteroid dihydrogenase enzyme.

In general, the present invention is directed to a composition for increasing the blood serum testosterone levels in a human, said composition comprising at least one prohormone and at least one anti-estrogen. More specifically, the invention is directed to a composition wherein the prohormone is selected from 19 Nor-4-androstene 3,17, dione; 4 Androstene-3,17, diol; 5-Androstene-3, 17 diol, Tribulus terrestris and mixtures thereof and wherein the anti-estrogen is selected from chrysin, indole-3-carbinol, saw palmetto extract and mixtures thereof. Yet more specifically, the invention is directed to oral dosage forms wherein the inventive compositions comprise enteric coatings, preferably at least three (3) enteric coatings.

Thus, there is disclosed an oral dosage delivery form adapted to increasing the testosterone levels in humans comprising:

a) a core comprising a mixture of at least one prohormone and at least one anti-estrogen in an amount sufficient to deliver a first portion of an effective amount of said mixture over the intended delivery time;

b) an enteric coating polymer over said core;

c) a coating of said prohormone/anti-estrogen mixture over said enteric polymer coating in an amount sufficient to deliver a second portion of an effective amount of said mixture over the intended delivery time;

d) a second enteric coating over said coating of said prohormone/anti-estrogen mixture; and

e) a third layer of said mixture over said second enteric polymer coating in an amount sufficient to deliver a third portion of an effective amount of said mixture over the intended delivery time.

The first, second, third and higher portions of the mixture can each independently range from 5 to about 50% of the effective amount of the mixture over the intended delivery time. Thus, for example, the core (first portion) may contain 25% of the dose for delivery to the large intestine/colon, the second portion may contain 25% for delivery to the small intestine and the third portion may contain the remainder (i.e., 50%) for delivery in the stomach.

The core may be formed around a biologically inert sphere such as a non-pareil wherein air suspension techniques are used to prepare the dosage forms. Non-pareil is known to those skilled in the art as a sugar particle that is widely used in the pharmaceutical industry. The core of the therapeutically active agent, the mixture of the prohormones and the anti-estrogen ingredients, may also contain other ingredients such as adhesives, anti-tack agents, binders, disintegrants, anti-foaming agents and lubricants. Especially preferred for use with the anabolic/anti-estrogen mixture of the invention is sodium lauryl sulfate. The presence of the sodium lauryl sulfate enhances the solubility of the formula. The inventive dosage forms may also be prepared through the use of know pelletization techniques. When using the pelletization technique, it is often advisable to include binders and processing aids in the therapeutically active mixture, such as dicalcium phosphate, magnesium stearate, stearic acid, silicon dioxide and the like. One skilled in the art will appreciate that the levels of materials, such as silicon dioxide, stearic acid, magnesium stearate, dicalcium phosphate and the like can range widely depending upon the physical characteristics of the active mixture and the parameters of the pelletizing machine. The enteric polymer coating may also contain components such as plasticizers and anti-tack agents.

It is preferred that a final protective coating be applied to the dosage forms according to the invention. This final protective coating should be a material that rapidly dissolves or disperses in the gastric juices.

As used herein and in the claims, the phrase “enteric polymer coating” means any coating that does not dissolve in the acidic environment of the stomach but does dissolve at a pH of 5 or higher. Representative enteric polymer coatings may be selected from the group consisting of ethylcellulose, hydroxypropylcellulose, hydroxypropylmethylcellulose, carboxymethylcellulose and mixtures thereof. Ethylcellulose is a common, microencapsular coating which will not readily dissolve or disperse in the stomach. Other aqueous or solvent based enteric coatings may be used as long as they do not readily dissolve or disperse in the gastric juices of the stomach but do dissolve or disperse in the intestinal fluid. Blends of various enteric polymers may also be used. For example, acrylic resins, shellac, wax or other film forming materials which will dissolve or disperse in the intestine but remain intact in the stomach, are possible alternatives. Most preferably, the enteric polymer coating comprises a water based emulsion polymer. A useful enteric coating is an ethylacrylate methacrylic acid copolymer sold under the trademark Eudragit® by Rhom GmbH of Domstadt, Germany. Representative Eudragit® coatings useful in the present invention include LD30, LD30-55, HP50, HP55, L-100 and mixtures thereof. One preferred enteric coating comprises a mixture of ethylcellulose, hydroxypropylmethylcellulose and polyethyleneglycol.

In the embodiment of the present invention, where there are two (2) or more coatings of enteric polymer separating three (3) or more doses of active, the first coating (closest to the core) is preferably an enteric coating that will survive until the dosage form arrives at the large intestine/colon. A preferred enteric coating is a series of methacrylic acid anionic copolymers known as Eudragit®S. These films are slowly soluble in the region of the digestive tract where the juices are neutral to weakly alkaline (i.e., the large intestine and the colon). Mixtures of these various enteric polymers recited above can be used in the present invention. Further, the use of plasticizers is preferred in the enteric polymer coatings.

The coating of the prohormone/anti-estrogen mixture over the enteric polymer may be identical to the composition of the core, except for the inert seed when air suspension techniques are used, or it may vary to some extent. It is preferred that the mixture remain the same, however, the disintegrants, lubricants, tackifying agents, partitioning agents, processing aids and the like may vary.

The low pH soluble protective coating may be any material that readily dissolves in the stomach fluids (pH of about 1.5 to 3) and provides protection to the underlying coating of the mixture. At least the protective coating will prevent abrasion to the prohormone/anti-estrogen mixture, reduce water absorption and reduce adhesion between individual dosage forms. Representative useful materials for the protective coating include Methocel® and other cellulosics and sugars that are water soluble. The low pH soluble protective coating may be omitted from the inventive dosage form, however, the preferred dosage form includes the protective coating.

The division of the prohormone/anti-estrogen mixture between numerous enteric coatings accomplishes a relatively consistent level of the actives in the blood serum. The division of the given dosages between the various enteric coatings can be controlled through the manufacturing process. Those skilled in the art will be able to adjust the air suspension of a fluidized bed, a rotor (a rotating disc) or a Wurster column device to accomplish the desired result. Spray rates through appropriate nozzles are also known to those proficient in the trade.

In the multiple enteric coating method described above, the final product beads can be incorporated into a double zero (00) or triple zero (000) gelatin capsule. Alternative methodologies of making the time released or controlled released dosage form of the invention include wet granulation, pelletization, encapsulation and inclusion in polymeric binders. For example, the active composition of the prohormone and the anti-estrogen compound can be mixed with various polymers, waxes, oils and the like and then fed to a granulating pellet mill. Various known apparatuses are known for preparing pellets and when in operation, the composition is fed from the hopper into the apparatus, humidified and heated and then continuously pushed into holes of a pellet die using rollers. The dosage forms are formed into hard pellets during passing through the holes of the die.

As a more specific example, the prohormone/anti-estrogen mixture according to the invention can be combined with the usual auxiliary agents and additives that also contain from about 30 to about 80% by weight based on the total weight of the composition of a combination of a water soluble and a water insoluble salt of casein. The dosage form of this type can then be treated in the usual way to provide a variety of solid dosage forms have a 2-phase matrix-controlled extended release profile. This process includes the employment of procedures known in the pharmaceutical industry, such as compressing, granulating, extruding, pelletizing and tableting in dry or wet manner. It is, of course, also possible to combine a variety of procedures to provide the desired product formulation in any one of various forms such as tablets, dragees, pellets, granules and the like. The amount of active substance present can be varied widely depending on the type of dosage form desired, for example, from 1% to 90%, based on the total weight of the pharmaceutical composition. More information on this form of the controlled release dosage form according to this invention can be found in U.S. Pat. No. 5,382,601.

Another process for preparing the dosage forms according to the invention is based on granules having an improved compatibility with the stomach, which comprises coating the carrier pellets or granules coated with xanthine, by means of an aqueous solution or dispersion of a gastric resistant coating material, separating the agglomerates obtained by sieving on an appropriate sieve, crushing them in a granulator and admixing the granules obtained, which are soluble or partially soluble in gastric juice to the granules coated with the gastric resistant material. More information on how to prepare the dosage forms according to the invention using this methodology can be found in U.S. Pat. No. 4,542,011.

A melt granulation material can also be used to prepare the dosage forms according to this invention. This method comprises mixing the active mixture with a binder having a melting point between about 40° and 100° C. and supplying energy to the mixture that will melt the binder and thereby granulate into pellets. These pellets are then cooled and pressed into a cohesive dosage form. Further details on this method of preparing the dosage forms according to the present invention can be found in U.S. Pat. No. 5,476,667.

The dosage forms according to the present invention may also be prepared by preparing a mixture of the active components combined with silicon dioxide, stearic acid, magnesium stearate and dicalcium phosphate. This mixture is then sprayed with a mixture of ethylcellulose polymer, hydroxypropylmethylcellulose and polyethylene glycol. A sufficient amount of this enteric coating is applied to the active mixture so as to form granules. These granules are then dried and then pressed into tablets using a tableting machine. The tablets are then preferably coated with an enteric coating as discussed above, or may have a protective coat applied as also discussed above. Information regarding useful tableting machines can be found in U.S. Pat. No. 5,910,324 and the references cited therein.

The invention will be better understood from the following Examples which are only representative of the invention as set forth in the claims.

### EXAMPLE 1

Using conventional equipment and techniques, the following compositions were prepared. Into a dry blender was added the following components to result in a 1.3 kg batch of a composition that would be wet granulated with the enteric coating.

| | | |
|---|---|---|
| 19 Nor-4-androstene 3,17 dione | 50 | gms |
| 4 Androstene-3,17 diol | 25 | gms |
| Tribulus terrestris (45% saponins by weight) | 125 | gms |
| Chrysin | 75 | gms |
| Indole-3-carbinol | 25 | gms |
| Saw palmetto extract | 90 | gms |
| Silicon dioxide | 13 | gms |
| Stearic acid | 19.5 | gms |
| Magnesium stearate | 17.55 | gms |
| Dicalcium phosphate | 809.95 | gms |
| TOTAL WEIGHT | 1.3 | Kg |

An enteric coating polymer solution was made up by mixing 80 gms of ethylcellulose polymer with 10 gms of hydroxypropylmethylcellulose and 10 gms of polyethylene glycol. This aqueous mixture was then applied to the androbolic/androgenic formula set out above under sufficient agitation and application rate as to form wet granules of the formula. The granules were then dried and fed to a 16 station Stokes B2 pelletizing machine. The punch size was 0.320 inches by 0.75 inches. The oblong pellets were prepared under a pressure of 22 tons per square inch.

### EXAMPLE 2

### Bioavailability Study

In this experiment, a comparative single dose, three-way cross over bioavailability study was conducted on the dosage form prepared in Example 1. The results from the bioavailability study will demonstrate that the dosage form according to the present invention produces blood serum testosterone levels that are more consistent over a 24 hour period than the intravenous administration of testosterone.

Industrial Applicability

While numerous products are offered to athletes to enhance their blood serum testosterone levels, none of them present a mixture of prohormones and anti-estrogen ingredients to ensure the safe elevation of testosterone levels without the serious side effects associated with testosterone conversion. Further, none of the prior art products have adapted the use of multiple enteric coatings which allow the mixture to be released over an extended period of time so as to achieve a sustained, elevated testosterone level. It is the application of the novel active ingredients and the use of multiple enteric coatings that represents a substantial advancement in the state of the art.

Having thus described the present invention in detail, it will be obvious to those skilled in the art that various changes or modifications may be made without departing from the scope of the invention defined in the appended claims and described in the specification.

I claim:

1. An oral dosage delivery form adapted to elevate the testosterone levels in a human, said dosage form comprising:
a) a core comprising a therapeutic agent, said therapeutic agent comprising:
i) at least one prohormone selected from the group consisting of 19 Nor-4-androstene-3,17 dione; 4-androstene-3β, 17β diol; 5-androstene-3β, 17β diol; Tribulus terrestris and mixtures thereof, and
ii) at least one anti-estrogen selected from the group consisting of chrysin, indole-3-carbinole, saw palmetto extract and mixtures thereof, wherein said core is in an amount sufficient to deliver a first portion of an effective amount of said therapeutic agent over the intended delivery time;
b) an enteric polymer coating over said core;
c) a coating of said therapeutic agent over said enteric polymer coating in an amount sufficient to deliver a second portion of an effective amount of said therapeutic agent over the intended delivery time;
d) a second enteric coating polymer coating over said second portion; and
e) a third portion of said therapeutic agent over said second enteric polymer coating, said third portion being an effective amount of said therapeutic agent over the intended delivery time.

2. The oral dosage delivery form according to claim 1 wherein said first portion is at least 10% of an effective amount of said therapeutic agent.

3. The oral dosage delivery form according to claim 1 wherein said second portion is at least 10% of an effective amount of said therapeutic agent.

4. The oral dosage delivery form according to claim 1 wherein said form additionally comprises a protective coating over said second portion of therapeutic agent.

5. The oral dosage delivery form according to claim 1 wherein said therapeutic agent is a mixture of 19 Nor-4-androstene-3,17 dione; 4-androstene-3β, 17β diol; 5-androstene-3, 17β diol; Tribulus terrestris; and mixtures thereof and at least one anti-estrogen selected from the group consisting of chrysin, indole-3-carbinole, saw palmetto extract and mixtures thereof.

6. The oral dosage delivery form according to claim 1 wherein said core additionally comprises at least one component selected from the group consisting of adhesives, anti-tack agents, disintegrants, anti-foam agents, lubricants and sodium lauryl sulfate.

7. The oral dosage delivery form according to claim 1 wherein enteric polymer coating additionally comprises at least one component selected from the group consisting of plasticizers and anti-tack agents.

8. The oral dosage delivery form according to claim 1 wherein said enteric polymer is selected from the group consisting of ethylcellulose, hydroxypropylcellulose, carboxymethylcellulose, acrylic resins, shellac, wax, ethylacrylate methacrylic acid copolymers and mixtures thereof.

9. The oral dosage delivery form according to claim 8 wherein said ethylacrylate methacrylic acid copolymer has a molecular weight of about 250,000.

10. The method of increasing testosterone levels in humans comprising the administration to said human of an effective amount of an oral dosage form according to claim 1.

11. A time-release oral dosage delivery form adapted to elevate the testosterone levels in a human, said dosage form comprising:
a) at least one prohormone selected from the group consisting of 19 Nor-4-Androstene 3,17 dione; 4-Androstene-3β,17β diol; 5-Androstene-3β,17β diol; Tribulus terrestris and mixtures thereof;
b) at least one anti-estrogen selected from the group consisting of chrysin, indole-3-carbinol, saw palmetto extract and mixtures thereof; and
c) an enteric coating.

12. The time-release oral dosage delivery form according to claim 11 wherein the enteric coating comprises ethylcellulose, hydroxypropylmethylcellulose and polyethylene glycol.

13. The time-release oral dosage delivery form according to claim 11 wherein said delivery form is prepared through wet granulation.

14. The time-release oral dosage delivery form according to claim 11 wherein said delivery form additionally comprises at least one component selected from the group consisting of silicon dioxide, stearic acid, magnesium stearate, dicalcium phosphate.

15. The time-release oral dosage delivery form according to claim 11 comprising:
a) 19 Nor-4-androstene 3,17 dione at a concentration of from 25 to 200 mgs;
b) 4 Androstene 3,17 diol at a concentration of from 25 to 200 mgs;
c) 5 Androstene 3,17 diol at a concentration of from 10 to 100 mgs;
d) Tribulus terrestris at a concentration of from 50 to 500 mgs;
e) chrysin at a concentration of from 30 to 300 mgs;
f) indole-3-carbinol at a concentration of from 10 to 100 mgs; and
g) saw palmetto extract at a concentration of from 40 to 400 mgs.

16. The time-release oral dosage delivery form according to claim 15 comprising:
a) 50 mgs of 19 Nor-45-androstene 3,17 dione;
b) 50 mgs of 4 Androstene-3,17 diol;
c) 25 mgs of 5 Androstene-3,17 diol;
d) 125 mgs of Tribulus Terrestris;
e) 75 mgs of chrysin;
f) 25 mgs of indole-3-carbinol; and
g) 90 mgs of saw palmetto extract.

17. The time-release oral dosage delivery form according to claim 15 wherein said enteric coating comprises ethylcellulose, hydroxypropylmethylcellulose and polyethylene glycol.
